(19) United States
(12) Patent Application Publication
(10) Pub. No.: US 2006/0014230 A1
(43) Pub. Date: Jan. 19, 2006

(54) Beta-lactamase detecting reagent composition, detection kit and detection method

(75) Inventor: Atsushi Murata, Kanagawa (JP)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Showa Yakuhin Kako Co., Ltd., Tokyo (JP)

(21) Appl. No.: 11/118,465

(22) Filed: Apr. 29, 2005

Related U.S. Application Data
(63) Continuation of application No. PCT/JP03/13835, filed on Oct. 29, 2003.

(30) Foreign Application Priority Data
Oct. 29, 2002 (JP) 2002-314681

(51) Int. Cl. C12Q 1/34; C07D 501/14

(52) U.S. Cl. 435/18; 540/222

(57) ABSTRACT

The present invention provides a reagent composition for detecting β-lactamase including as a β-lactamase detection substrate 3-[2,4-dinitrostyryl]-7-(2-thienylacetamido]-3-cephem-4-carboxylic acid, or 7-[2-(2-aminothiazol-4-yl)-2-(1-carboxy-1-methylethoxy-imino)acetamido]-3-(2,4-dinitrostyryl)-3-cephem-4-carboxylic acid, and at least one β-lactamase inhibitor selected from the group consisting of clavulanic acid, aztreonam, ethylenediaminetetraacetic acid, and cloxacillin, which composition can detect β-lactamases rapidly and easily with high sensitivity. The present invention also provides a detection kit including the detecting reagent composition. Further, the present invention provides a β-lactamase detection method where a liquid specimen containing a target substance to be analyzed is brought into contact with the composition.

### TECHNICAL FIELD

The present invention relates to a reagent composition for detecting β-lactamase, a kit for detecting β-lactamase and a β-lactamase detection method.

### BACKGROUND ART

β-lactamases, which are a family of enzymes that hydrolyze the β-lactam ring contained in a molecule of β-lactam based antibacterial agents, are classified into class A, B, C and D according to amino-acid sequence homology in the enzyme proteins. Extended-spectrum β-lactamase (ESBL) is an enzyme that has a more extended range of substrates (i.e., β-lactam based antibacterial agents) to be decomposed by the enzyme as compared with the conventional class A β-lactamase. These β-lactamases deactivate the antibacterial action of the β-lactam based antibacterial agents. Particularly, the ESBL has been recognized as a cause of nosocomial infection and perceived as a serious problem. Administration of β-lactam based antibacterial agents against the bacteria having developed a resistance to the β-lactam based antibacterial agents not only would be a hopeless cure, but also might lead to spreading of new resistant bacteria. Thus, it is necessary to detect the β-lactamases rapidly and accurately in determining a sure cure.

The methods currently used for detecting the β-lactamases are roughly divided into the following four methods: (1) chromogenic cephalosporin method, (2) acidmetry method, (3) iodometry method, and (4) UV method. In addition, there can be employed a cultivation method for comparing the minimum inhibitory concentrations (MIC). Principal among the commercially available products for detecting the β-lactamases are: a product capable of indicating whether β-lactamase is present or absent using the chromogenic cephalosporin method; a product capable of detecting the β-lactamases belonging to the class A and class C using the acidmetry method; a product capable of detecting the class B β-lactamase or ESBL using the cultivation method, and the like.

The chromogenic cephalosporin method uses cephalosporin that will cause a color change upon the cleavage of its β-lactam ring by the application of β-lactamase thereto. This method has the advantage that the detection sensitivity is excellent because the reagent itself results in a color change. However, the conventional commercially available products using the chromogenic cephalosporin method employ as a detection substrate nitrocefin (i.e., 3-[2,4-dinitrostyryl]-7-(2-thienylacetamido)-3-cephem-4-carboxylic acid in Japanese Patent Unexamined Publication (JP Kokai) Sho 48-50787). This product cannot react to all the classes of β-lactamases and cannot distinguish one class from the others, although the detection can be achieved in a short period of time, i.e., about 30 minutes. Further, among the products based on the chromogenic cephalosporin method, no product is conventionally known that is characterized by using nitrocefin in combination with a β-lactamase inhibitor.

A novel compound capable of detecting the ESBL based on the chromogenic cephalosporin method is disclosed in PCT pamphlet WO 02/24707. However, this PCT pamphlet does not disclose the combined use of the compound and a particular β-lactamase inhibitor, nor suggest the possibility of detecting any classes of β-lactamases in addition to the ESBL.

### DISCLOSURE OF INVENTION

An object of the present invention is to provide a reagent composition for detecting β-lactamase, a kit for detecting β-lactamase and a β-lactamase detection method, which allow rapid detection of the β-lactamases.

The inventors of the present invention have found that the above-mentioned object can be achieved by the combined use of a particular β-lactamase detection substrate and a β-lactamase inhibitor, in particular, a combination of two or three kinds of particular β-lactamase inhibitor components.

Namely, the present invention provides a reagent composition for detecting β-lactamase for use with a chromogenic cephalosporin method, comprising a β-lactamase detection substrate and a β-lactamase inhibitor.

The present invention also provides a kit for detecting β-lactamase comprising one or more detecting reagent compositions selected from the group consisting of (a) to (e) shown below:

(a) a detecting reagent composition comprising a compound represented by general formula (1) or physiologically acceptable salt thereof as a β-lactamase detection substrate, and

a combination of aztreonam and ethylenediaminetetraacetic acid as a β-lactamase inhibitor;

(b) a detecting reagent composition comprising a compound represented by general formula (1) or physiologically acceptable salt thereof as a β-lactamase detection substrate, and

a combination of clavulanic acid and ethylenediaminetetraacetic acid as a β-lactamase inhibitor;

(c) a detecting reagent composition comprising a compound represented by general formula (1) or physiologically acceptable salt thereof as a β-lactamase detection substrate, and

a combination of aztreonam and clavulanic acid as a β-lactamase inhibitor;

(d) a detecting reagent composition comprising a compound represented by general formula (2) or carboxylate derivative thereof as a β-lactamase detection substrate, and

a combination of aztreonam and ethylenediaminetetraacetic acid as a β-lactamase inhibitor; and

(e) a detecting reagent composition comprising a compound represented by general formula (2) or carboxylate derivative thereof as a β-lactamase detection substrate, and

a combination of aztreonam, clavulanic acid, and ethylenediaminetetraacetic acid as a β-lactamase inhibitor.

The present invention also provides a kit for detecting β-lactamase comprising one or more detecting reagent compositions selected from the group consisting of (f), (g), (h) and (i) shown below:

(f) a detecting reagent composition comprising a compound represented by general formula (1) or physiologically acceptable salt thereof as a β-lactamase detection substrate, and

aztreonam as a β-lactamase inhibitor;

(g) a detecting reagent composition comprising a compound represented by general formula (1) or physiologically acceptable salt thereof as a β-lactamase detection substrate, and

clavulanic acid as a β-lactamase inhibitor;

(h) a detecting reagent composition comprising a compound represented by general formula (2) or carboxylate derivative thereof as a β-lactamase detection substrate, and

aztreonam as a β-lactamase inhibitor; and

(i) a detecting reagent composition comprising a compound represented by general formula (2) or carboxylate derivative thereof as a β-lactamase detection substrate, and

a combination of aztreonam and clavulanic acid as a β-lactamase inhibitor.

In addition, the present invention provides a β-lactamase detection method comprising the step of bringing a liquid specimen containing a target substance to be analyzed in contact with the above-mentioned reagent composition for detecting β-lactamase.

Further, the present invention provides a β-lactamase detection method, comprising the step of bringing a liquid specimen containing a target substance to be analyzed into contact with the above-mentioned reagent composition for detecting β-lactamase held in a sample holding portion of a kit for detecting β-lactamase.

The present invention also provides a β-lactamase detection method, comprising the step of bringing a liquid specimen containing a target substance to be analyzed into contact with a reagent composition for detecting β-lactamase held in a sample holding portion of the above-mentioned kit for detecting β-lactamase .

### BRIEF DESCRIPTION OF DRAWING

FIG. 1 is a schematic perspective view showing one embodiment of the kit for detecting β-lactamase according to the present invention.

### BEST MODE FOR CARRYING OUT THE INVENTION

The present invention is based on the chromogenic cephalosporin method.

A compound represented by the following general formula (1) or physiologically acceptable salts thereof disclosed in JP Kokai Sho 48-50787, or a compound represented by the following general formula (2) or carboxylate derivatives thereof disclosed in WO 02/24707, which patent literatures are incorporated herein in their entirety by reference, may be used as the substrate for detecting the β-lactamases for use in the present invention.

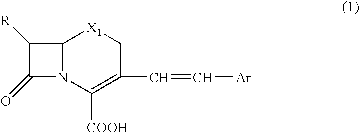

In general formula (1), R is a formamide group or a group represented by formula of R^{u}CH₂CONH, R^{u}OCH₂CONH, R^{u}CONH, R^{u}CH(NH₂)CONH or R^{u}C(═NOH)CONH, wherein R^{u} is thienyl group, phenyl group or naphthyl group; Ar is a phenyl group having a substitution of cyano group or nitro group at the 2-, 4-, or 2,4-position; and —X₁— is —S— or —SO—.

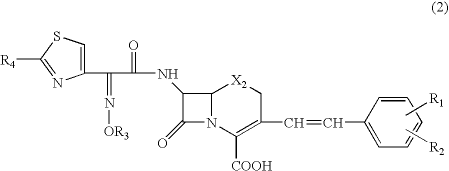

In general formula (2), R₁ and R₂, which may be the same or different, are each hydrogen atom, nitro group, or cyano group; R₃ is an alkyl group having 1 to 6 carbon atoms which may have a substituent of carboxyl group; R₄ is hydrogen atom or amino group; and —X₂— is —S— or —SO—, provided that R₁ and R₂ do not represent hydrogen atom at the same time.

The compound represented by general formula (1), serving as the β-lactamase detection substrate may preferably be 3-[2,4-dinitrostyryl]-7-(2-thienylacetamido)-3-cephem-4-carboxylic acid or physiologically acceptable salts thereof.

Preferable compounds represented by general formula (2) include a compound wherein —X₂— is —S—; a compound wherein R₃ is methyl group which may be substituted with carboxyl group or propyl group substituted with carboxyl group; a compound wherein R₄ is amino group; and a compound wherein —X₂— is —S—, R₃ is methyl group which may be substituted with carboxyl group or propyl group substituted with carboxyl group, and R₄ is amino group.

The following compounds and carboxylate derivatives thereof are preferable:

7-[2-(2-aminothiazol-4-yl)-2-(1-carboxy-1-methylethoxy-imino)acetamido]-3-(2,4-dinitrostyryl)-3-cephem-4-carboxylic acid,

7-[2-(2-aminothiazol-4-yl)-2-(1-carboxy-1-methylethoxy-imino)acetamido]-3-(2,6-dinitrostyryl)-3-cephem-4-carboxylic acid,

7-[2-(2-aminothiazol-4-yl)-2-(1-carboxy-1-methylethoxy-imino) acetamido]-3-(4-nitrostyryl)-3-cephem-4-carboxylic acid,

7-[2-(2-aminothiazol-4-yl)-2-(1-carboxy-1-methylethoxy-imino)acetamido]-3-(2,4-dicyanostyryl)-3-cephem -4-carboxylic acid,

7-[2-(2-aminothiazol-4-yl)-2-(1-carboxy-1-methylethoxy-imino)acetamido]-3-(4-cyanostyryl)-3-cephem-4-carboxylic acid,

7-[2-(2-aminothiazol-4-yl)-2-(1-carboxy-1-methylethoxy-imino)acetamido]-3-(2-cyanostyryl)-3-cephem-4-carboxylic acid,

7-[2-(1-carboxy-1-methylethoxyimino)-2-(thiazol-4-yl)acetamido]-3-(2,4-dinitrostyryl)-3-cephem -4-carboxylic acid,

7-[2-(2-aminothiazol-4-yl)-2-(1-carboxy-1-methylethoxy-imino)acetamido]-3-(2,4-dinitrostyryl)-3-cephem-4-carboxylic acid-1-oxide,

7-[2-(2-aminothiazol-4-yl)-2-methoxyiminoacetamido]-3-(4-nitrostyryl)-3-cephem-4-carboxylic acid,

7-[2-(2-aminothiazol-4-yl)-2-methoxyiminoacetamido]-3-(2,4-dicyanostyryl)-3-cephem-4-carboxylic acid,

7-[2-(2-aminothiazol-4-yl)-2-methoxyiminoacetamido]-3-(2,6 -dicyanostyryl)-3-cephem-4-carboxylic acid,

7-[2-(2-aminothiazol-4-yl)-2-methoxyiminoacetamido]-3-(2-cyanostyryl)-3-cephem-4-carboxylic acid,

7-[2-(2-aminothiazol-4-yl)-2-carboxymethoxyimino-acetamido]-3-(2,4-dinitrostyryl)-3-cephem-4-carboxylic acid,

7-[2-(2-aminothiazol-4-yl)-2-carboxymethoxyimino-acetamido]-3-(2,6-dinitrostyryl)-3-cephem-4-carboxylic acid,

7-[2-(2-aminothiazol-4-yl)-2-carboxymethoxyimino-acetamido]-3-(4-nitrostyryl)-3-cephem-4-carboxylic acid,

7-[2-(2-aminothiazol-4-yl)-2-carboxymethoxyimino-acetamido]-3-(2-nitrostyryl)-3-cephem-4-carboxylic acid,

7-[2-(2-aminothiazol-4-yl)-2-carboxymethoxyimino-acetamido]-3-(2,4-dicyanostyryl)-3-cephem-4-carboxylic acid,

7-[2-(2-aminothiazol-4-yl)-2-carboxymethoxyimino-acetamido]-4-(4-cyanostyryl)-3-cephem-4-carboxylic acid,

7-[2-(2-aminothiazol-4-yl)-2-carboxymethoxyimino-acetamido]-4-(2-cyanostyryl)-3-cephem-4-carboxylic acid,

7-[2-carboxymethoxyimino-2-(thiazol-4-yl)acetamido]-3-(2,4-dinitrostyryl)-3-cephem-4-carboxylic acid, and

7-[2-(2-aminothiazol-4-yl)-2-carboxymethoxyimino-acetamido]-3-(2,4-dinitrostyryl)-3-cephem-4-carboxylic acid-1-oxide.

In particular, the following compounds and carboxylate derivatives thereof are preferable:

7-[2-(2-aminothiazol-4-yl)-2-(1-carboxy-1-methylethoxy-imino)acetamido]-3-(2,4-dinitrostyryl)-3-cephem-4-carboxylic acid,

7-[2-(2-aminothiazol-4-yl)-2-(1-carboxy-1-methylethoxy-imino)acetamido]-3-(4-nitrostyryl)-3-cephem-4-carboxylic acid,

7-[2-(2-aminothiazol-4-yl)-2-(1-carboxy-1-methylethoxy-imino) acetamido]-3-(4-cyanostyryl)-3-cephem-4-carboxylic acid,

7-[2-(2-aminothiazol-4-yl)-2-methoxyiminoacetamido]-3-(4-nitrostyryl)-3-cephem-4-carboxylic acid,

7-[2-(2-aminothiazol-4-yl)-2-carboxymethoxyimino-acetamido]-3-(2,4-dinitrostyryl)-3-cephem-4-carboxylic acid, and

7-[2-(2-aminothiazol-4-yl)-2-carboxymethoxyimino-acetamido]-3-(4-nitrostyryl)-3-cephem-4-carboxylic acid.

In particular, 7-[2-(2-aminothiazol-4-yl)-2-(1-carboxy-1-methylethoxy-imino)acetamido]-3-(2,4-dinitrostyryl)-3-cephem-4-carboxylic acid or 7-[2-(2-aminothiazol-4-yl)-2-carboxymethoxyiminoacetamido]-3-(2,4-dinitrostyryl)-3-cephem-4-carboxylic acid is preferable.

The β-lactamase inhibitor for use in the present invention is not particularly limited so long as it is a compound having β-lactamase inhibitory activity. As the β-lactamase inhibitor, at least one selected from the group consisting of clavulanic acid or compounds that can work equivalently thereto, aztreonam, cloxacillin, a chelating agent capable of chelation of zinc ion, sulbactam, tazobactam, thienamycin and carumonam may preferably be used. Examples of the chelating agent capable of chelation of zinc ion are ethylenediaminetetraacetic acid (EDTA), cyclohexanediaminetetraacetic acid and mercapto compounds. The mercapto compounds, for example, compounds disclosed in JP Kokai 2000-224998 and 2001-299388, can be used. To be more specific, there can be employed sulfur-containing compounds, such as mercaptoacetic acid, mercaptopropionic acid, in particular, 2-mercaptopropionic acid, and sodium salts, potassium salts, calcium salts, barium salts or quaternary ammonium salts thereof; and organic thiol compounds, e.g., mercaptoethanol and the like. Particularly preferred as the chelating agent capable of chelation of zinc ion is EDTA.

In particular, it is preferable that the β-lactamase inhibitor be selected from the group consisting of clavulanic acid; aztreonam; a combination of aztreonam and ethylenediaminetetraacetic acid; a combination of clavulanic acid and ethylenediaminetetraacetic acid; a combination of aztreonam and clavulanic acid; and a combination of aztreonam, clavulanic acid, and ethylenediamine-tetraacetic acid.

Examples of the combined use of the β-lactamase detection substrate and the β-lactamase inhibitor, preferably employed in the present invention are as follows:

a detecting reagent composition comprising a compound represented by the above-mentioned general formula (1) or physiologically acceptable salt thereof as the β-lactamase detection substrate, and at least one β-lactamase inhibitor selected from the group consisting of clavulanic acid, aztreonam, ethylenediaminetetraacetic acid, and cloxacillin; and

a detecting reagent composition comprising a compound represented by the above-mentioned general formula (2) or carboxylate derivative thereof as the β-lactamase detection substrate, and at least one β-lactamase inhibitor selected from the group consisting of clavulanic acid, aztreonam, ethylenediaminetetraacetic acid, and cloxacillin.

In addition, a kit for detecting β-lactamase including the above-mentioned composition (c), and further one or more compositions selected from the group consisting of (f) through (i) shown below is also preferable:

(f) a detecting reagent composition comprising a compound represented by general formula (1) or physiologically acceptable salt thereof as the β-lactamase detection substrate, and aztreonam as the β-lactamase inhibitor;

(g) a detecting reagent composition comprising a compound represented by general formula (1) or physiologically acceptable salt thereof as the β-lactamase detection substrate, and clavulanic acid as the β-lactamase inhibitor;

(h) a detecting reagent composition comprising a compound represented by general formula (2) or carboxylate derivative thereof as the β-lactamase detection substrate, and aztreonam as the β-lactamase inhibitor; and

(i) a detecting reagent composition comprising a compound represented by general formula (2) or carboxylate derivative thereof as the β-lactamase detection substrate, and a combination of aztreonam and clavulanic acid as the β-lactamase inhibitor.

In the detecting reagent composition of the present invention, the ratio by mass of the β-lactamase detection substrate to the β-lactamase inhibitor may preferably be in the range of (1:10) to (100:1), more preferably in the range of (1:1) to (50:1), and further preferably in the range of (1:1) to (1:10).

The detecting reagent composition of the present invention may be prepared in the form of a solution by dissolving the β-lactamase detection substrate and the β-lactamase inhibitor in a solvent, such as water, phosphate buffer solution, dimethyl sulfoxide, or a mixture thereof. Of those solvents, a mixture of dimethyl sulfoxide and phosphate buffer solution is especially preferable, and in this case, the mixing ratio is not particularly limited. To be more specific, the β-lactamase detection substrate and the β-lactamase inhibitor may be dissolved in a solvent so that the concentration of the β-lactamase detection substrate may preferably be in the range of 0.025 to 62.5 mg/mL, more preferably 1.25 to 12.5 mg/mL, and that of the β-lactamase inhibitor may preferably be in the range of 0.0125 to 12.5 mg/mL, more preferably 1.25 to 12.5 mg/mL, thereby obtaining a solution containing a reagent composition for detecting β-lactamase.

It is preferable to use the detecting reagent composition of the present invention adjusted to have pH 6.0 to 8.0. The detecting reagent composition may preferably be adjusted to about pH 5.5 to 8.5 in the case where the compound represented by the aforementioned general formula (1) is used as the β-lactamase detection substrate. When the compound represented by the aforementioned general formula (2) is used as the β-lactamase detection substrate, the detecting reagent composition may preferably be adjusted to about pH 6.0 to 8.0.

The solution containing detecting reagent composition thus prepared may further comprise polymers or the like, such as polyvinylpyrrolidone, hydroxymethyl cellulose, polyvinyl chloride, and methacrylic acid copolymer in such an amount that may not hinder the effects of the present invention. Addition of such polymers or the like is advantageous because the solution containing the detecting reagent composition of the present invention becomes more stable. Of those polymers particularly preferable is methacrylic acid copolymer.

The specimen used in the present invention can be collected, for example, from pharynx and nasal cavity of patients with a cotton swab, and in addition, from urine, sputum, pus and the like. The specimen may be prepared in a liquid form by dissolving the specimen in a -solvent such as water, phosphate buffer solution, physiological saline solution or the like. Preferably, the liquid specimen may be prepared to have a bacteria concentration of 10⁴ to 10¹⁰ CFU/mL.

The liquid specimen thus prepared may further contain oligosaccharide, surfactant, dextrin, and the like in such amounts that may not impair the effects of the present invention.

The kit for detecting β-lactamase of the present invention may be in any form so long as the above-mentioned detecting reagent composition is contained therein, for example, in the form of a disk, combination of a disk and a board, a plate, a strip or the like. The form preferably used is a disk-type one.

When necessary, the kit of the present invention may include a positive control liquid made of a solution containing β-lactamase and the detecting reagent composition of the present invention, and a negative control liquid made of a solution containing the β-lactamase detection substrate.

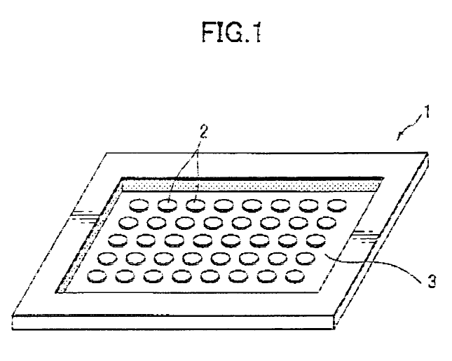

FIG. 1 is a schematic perspective view showing a kit for detecting β-lactamases according to the present invention, but the present invention is not limited thereto. Referring to FIG. 1, a detection kit according to the present invention includes a sample container 1, and a base member 3 which is stored in the container and bears a plurality of sample holding portions 2 thereon. The sample holding portion 2 has an opening on which a prepared sample (a liquid specimen or control liquid) is dropped. The materials for the sample container, base member, and the sample holding portion, which constitute the detection kit of the present invention, are not particularly limited. There is no restraint on the size and the shape of such parts.

One of the preferable kits according to the present invention is the one that contains the above-mentioned detecting reagent composition (c) as an essential composition, and further contains one or more detecting reagent compositions selected from the group consisting of the compositions (a), (b), (d) and (e).

Also, the kit for detecting β-lactamase that contains the detecting reagent composition (d) as an essential composition, and further contains one or more detecting reagent compositions selected from the group consisting of the compositions (a), (b), (c) and (e) is preferred.

In particular, the kit for detecting β-lactamase that contains all the above-mentioned detecting reagent compositions (a) through (e) is preferable.

In addition, preferably employed is the kit according to the present invention that contains one or more detecting reagent compositions selected from the group consisting of the compositions (c) and f) through (i).

More preferably used is the kit according to the present invention that contains the above-mentioned detecting reagent composition (c) as an essential composition, and further contains one or more detecting reagent compositions selected from the group consisting of the compositions (f), (g), (h) and (i).

Also, preferably used is the kit for detecting β-lactamase according to the present invention that contains the above-mentioned detecting reagent composition (h) as an essential composition, and further contains one or more detecting reagent compositions selected from the group consisting of the compositions (c), (f), (g) and (i).

In particular, the kit for detecting β-lactamase that contains all the above-mentioned detecting reagent compositions (c) and (f) through (i) is further preferable.

In the detection kit of the present invention, it is most preferable that 3-[2,4-dinitrostyryl]-7-(2-thienylacetamido]-3-cephem-4-carboxylic acid or 7-[2-(2-aminothiazol-4-yl)-2-(1-carboxy-1-methylethoxy-imino)acetamido]-3-(2,4-dinitrostyryl)-3-cephem-4-carboxylic acid (hereinafter referred to as “HMRZ compound”) be contained as the substrate for detecting the β-lactamases in the above-mentioned detecting reagent compositions (a) to (i).

By using the kit according to the present invention, the class A, B, C and D β-lactamases, and ESBL can be detected rapidly by one operation. Therefore, use of the kit according to the present invention will lead to choice of a proper antibacterial agent, which can further enhance the effects of cure.

The present invention also relates to a β-lactamases detection method, comprising the step of adding a liquid specimen containing a target substance to be analyzed to a solution containing any of the above-mentioned reagent composition for detecting β-lactamase s.

The method of the present invention includes the step of adding a liquid specimen containing a target substance to be analyzed to the solution containing the reagent composition for detecting β-lactamase prepared in such a manner as described above. It is possible to discriminate between positive and negative by checking whether the color tone is changed from yellow to red, or not, after a lapse of a given time, for example, about 30 minutes, at a predetermined temperature, for example, at room temperature. Further, the activity assay can also be performed by measuring a change in color tone at a particular wavelength.

By using the method of the present invention, it is possible to rapidly detect β-lactamases in urine and sputum of patients with diseases, for example, caused by β-lactamase producing bacteria, such as urethral infection and pneumonia, and nosocomial infections derived from Klebsiella pneumoniae and Escherichia coli.

### EXAMPLES

1. Preparation of Solution Containing Reagent Composition for Detecting β-Lactamase

1.25 mg of a β-lactamase detection substrate and 1.25 mg of a β-lactamase inhibitor, both of which are shown in the following Table 1, were dissolved in a mixed solvent of 0.1 mL of dimethyl sulfoxide and 0.9 mL of a phosphate buffer solution, to prepare a solution containing reagent composition for detecting β-lactamase.

TABLE 1

| Reagent | B-lactamase detection | |
|---|---|---|
| No. | substrate | B-lactamase inhibitor |
| 1 | nitrocefin | — |
| 2 | nitrocefin | AZT, EDTA |
| 3 | nitrocefin | CVA, EDTA |
| 4 | nitrocefin | AZT, CVA |
| 5 | HMRZ compound | AZT, EDTA |
| 6 | HMRZ compound | AZT, CVA, EDTA |
| 7 | HMRZ compound | — |
| 8 | nitrocefin | AZT |
| 9 | nitrocefin | CVA |
| 10 | nitrocefin | AZT, CVA |
| 11 | HMRZ compound | AZT |
| 12 | NMRZ compound | AZT, CVA |

In the above table,

nitrocefin denotes 3-[2,4-dinitrostyryl]-7-(2-thienylacetamido]-3-cephem-4-carboxylic acid,

HMRZ compound denotes 7-[2-(2-aminothiazol-4-yl)-2-(1-carboxy-1-methylethoxy-imino)acetamido]-3-(2,4-dinitrostyryl)-3-cephem-4-carboxylic acid,

AZT denotes aztreonam, EDTA denotes ethylenediaminetetraacetic acid, and CVA denotes clavulanic acid.
2. Preparation of Plate

The obtained solution containing reagent composition for detecting β-lactamase was applied to a microplate so that the amounts of the β-lactamase detection substrate and the β-lactamase inhibitor might individually be 12.5 μg per well.
3. Preparation of Liquid Specimen Containing Target Substance to be Analyzed

Using β-lactamase nonproducing bacteria, class-A β-lactamase-producing bacteria, class-B β-lactamase-producing bacteria, class-C β-lactamase-producing bacteria, and ESBL-producing bacteria, shown in the following Table 2, each of liquid specimens was prepared by inoculating each kind of bacteria into a phosphate buffer solution to have a concentration of 10⁸ CFU/mL.

TABLE 2

| Produced Enzyme | Name of Bacteria |
|---|---|
| Enzyme non-produced | S. aureus |
| Class A | K. pneumoniae |
| Class B | S. marcescens |
| Class C | S. marcescens |
| ESBL | E. coli |

4. Reactivities to β-Lactamases

The liquid specimen was inoculated into the microplate prepared in the above in an amount of 50 μL/well. The reactivities to the β-lactamases were examined by observing a change in color tone 30 minutes after inoculation.

TABLE 3
Reactivities to β-lactamases

| Produced | Reagent No. | | | | | | | | | | | |
|---|---|---|---|---|---|---|---|---|---|---|---|---|
| Enzyme | 1 | 2 | 3 | 4 | 5 | 6 | 7 | 8 | 9 | 10 | 11 | 12 |
| Non- | − | − | − | − | − | − | − | − | − | − | − | − |
| produced | | | | | | | | | | | | |
| Class A | + | + | − | − | − | − | − | + | − | − | − | − |
| Class B | + | − | − | + | − | − | ± | + | + | + | ± | ± |
| Class C | + | − | + | − | − | − | ± | − | + | − | − | − |
| ESBL | + | + | − | − | + | − | ± | + | − | − | + | − |

+: positive (The color changed from yellow to red.)
−: negative (The yellow color was not changed.)
±: (A slight change was observed.)

According to the present invention, β-lactamases can be detected rapidly and easily with high sensitivity. In addition, the combined use of a particular detection substrate and a particular inhibitor can make it possible to detect even the class of β-lactamases, with a slight detection error in the present invention. Rapid detection of the class can lead to choice of a proper antibacterial agent, which provides a highly effective cure.

1. A reagent composition for detecting β-lactamase for use with a chromogenic cephalosporin method, comprising a β-lactamase detection substrate and a β-lactamase inhibitor.

2. The reagent composition for detecting β-lactamase as claimed in claim 1, wherein the β-lactamase detection substrate is a compound represented by general formula (1) or physiologically acceptable salt thereof, or a compound represented by general formula (2) or carboxylate derivative thereof: 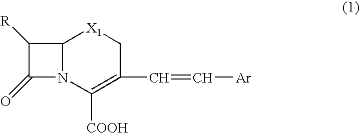
wherein R is a formamide group or a group represented by formula of R^{u}CH₂CONH, R^{u}OCH₂CONH, R^{u}CONH, R^{u}CH(NH₂)CONH, or R^{u}C(═NOH)CONH, wherein R^{u} is a thienyl group, phenyl group or naphthyl group: Ar is a phenyl group having a substitute of cyano group or nitro group at the 2-, 4-, or 2,4-position; and —X₁—is—S— or —SO—; 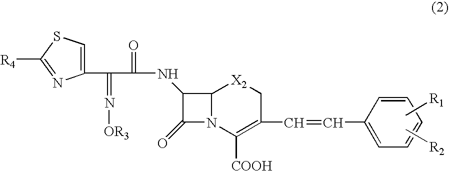
wherein R₁ and R₂, which may be same or different, are each hydrogen atom, nitro group, or cyano group; R₃ is alkyl group having 1 to 6 carbon atoms which may have a substituent of carboxyl group; R₄ is hydrogen atom or amino group; and —X₂— is —S— or —SO—, provided that R₁ and R₂ do not represent hydrogen atom at the same time.

3. The reagent composition for detecting β-lactamase as claimed in claim 1, wherein the β-lactamase inhibitor is at least one selected from the group consisting of clavulanic acid and compounds that can work equivalently thereto, aztreonam, cloxacillin, chelating agent capable of chelation of zinc ion, sulbactam, tazobactam, thienamycin and carumonam.

4. The reagent composition for detecting β-lactamase as claimed in claim 3, therein the β-lactamase inhibitor is selected from the group consisting of clavulanic acid; aztreonam; a combination of aztreonam and ethylenediaminetetraacetic acid; a combination of clavulanic acid and ethylenediaminetetraacetic acid; a combination of aztreonam and clavulanic acid; and a combination of aztreonam, clavulanic acid, and ethylenediaminetetraacetic acid.

5. A kit for detecting β-lactamase comprising one or more detecting reagent composition selected from the group consisting of:
(a) a detecting reagent composition comprising as a β-lactamase detection substrate the compound represented by general formula (1) or physiologically acceptable salt thereof as claimed in claim 2, and as a β-lactamase inhibitor a combination of aztreonam and ethylenediaminetetraacetic acid;
(b) a detecting reagent composition comprising as a β-lactamase detection substrate the compound represented by general formula (1) or physiologically acceptable salt thereof as claimed in claim 2, and as a β-lactamase inhibitor a combination of clavulanic acid and ethylenediaminetetraacetic acid;
(c) a detecting reagent composition comprising as a β-lactamase detection substrate the compound represented by general formula (1) or physiologically acceptable salt thereof as claimed in claim 2, and as a β-lactamase inhibitor a combination of aztreonam and clavulanic acid;
(d) a detecting reagent composition comprising as a β-lactamase detection substrate the compound represented by general formula (2) or carboxylate derivative thereof as claimed in claim 2, and as β-lactamase inhibitor a combination of aztreonam and ethylenediaminetetraacetic acid; and
(e) a detecting reagent composition comprising as a β-lactamase detection substrate the compound represented by general formula (2) or carboxylate derivative thereof as claimed in claim 2, and as a β-lactamase inhibitor a combination of aztreonam, clavulanic acid, and ethylenediaminetetraacetic acid.

6. The kit for detecting a β-lactamase as claimed in claim 5, comprising as an essential composition the detecting reagent composition (c), and further comprising one or more detecting reagent compositions selected from the group consisting of the detecting reagent compositions (a), (b), (d) and (e).

7. The kit for detecting β-lactamase as claimed in claim 5, comprising as an essential composition the detecting reagent composition (d), and further comprising one or more detecting reagent compositions selected from the group consisting of the detecting reagent compositions (a), (b), (c), and (e).

8. The kit for detecting β-lactamase as claimed in claim 5, comprising all the detecting reagent compositions (a) through (e).

9. A kit for detecting β-lactamase comprising one or more detecting reagent composition selected from the group consisting of:
(f) a detecting reagent composition comprising as a β-lactamase detection substrate the compound represented by general formula (1) or physiologically acceptable salt thereof as claimed in claim 2, and as a β-lactamase inhibitor aztreonam;
(g) a detecting reagent composition comprising as a β-lactamase detection substrate the compound represented by general formula (1) or physiologically acceptable salt thereof as claimed in claim 2, and as β-lactamase inhibitor clavulanic acid;
(h) a detecting reagent composition comprising as a β-lactamase detection substrate the compound represented by general formula (2) or carboxylate derivative thereof as claimed in claim 2, and as a β-lactamase inhibitor aztreonam; and
(i) a detecting reagent composition comprising as a β-lactamase detection substrate the compound represented by general formula (2) or carboxylate derivative thereof as claimed in claim 2, and as a β-lactamase inhibitor a combination of aztreonam and clavulanic acid.

10. The kit for detecting β-lactamase as claimed in claim 9, further comprising a detecting reagent composition comprising as a β-lactamase detection substrate the compound represented by general formula (1) or physiologically acceptable salt thereof, and as a β-lactamase inhibitor a combination of aztreonam and clavulanic acid.

11. The kit for detecting β-lactamase as claimed in claim 9, comprising the detecting reagent composition (h), and further comprising one or more detecting reagent compositions selected from the group consisting of the detecting reagent compositions (f), (g), and (i).

12. The kit for detecting β-lactamase as claimed in claim 10, comprising all the detecting reagent compositions (f) through (i).

13. A β-lactamase detection method, comprising:
bringing a liquid specimen containing a target substance to be analyzed into contact with the reagent composition for detecting β-lactamase as claimed in claim 1.

14. A β-lactamase detection method, comprising:
bringing a liquid specimen containing a target substance to be analyzed into contact with the reagent composition for detecting β-lactamase as claimed in claim 1 held in a sample holding portion of a kit for detecting β-lactamase.

15. A β-lactamase detection method, comprising:
bringing a liquid specimen containing a target substance to be analyzed into contact with a reagent composition for detecting β-lactamase held in a sample holding portion of the kit for detecting β-lactamase as claimed in claim 5.
